(19) United States
(12) Patent Application Publication
(10) Pub. No.: US 2024/0009171 A1
(43) Pub. Date: Jan. 11, 2024

(54) COMPOSITIONS AND METHODS FOR DELIVERY OF PSILOCIN AND PRODRUGS THEREOF

(71) Applicant: Callitas Health Inc., Naples, FL (US)

(72) Inventor: Joshua G. Maurice, Sarasota, FL (US)

(21) Appl. No.: 18/470,697

(22) Filed: Sep. 20, 2023

Related U.S. Application Data
(63) Continuation of application No. 17/720,501, filed on Apr. 14, 2022.
(60) Provisional application No. 63/175,890, filed on Apr. 16, 2021.

(51) Int. Cl. A61K 31/4045; A61K 47/18; A61K 47/10; A61K 9/00

(52) U.S. Cl. CPC A61K 31/4045; A61K 47/183; A61K 47/10; A61K 9/006

(57) ABSTRACT

A system and method for delivering psilocin or a prodrug of psilocin to a mammal. The system includes a water-soluble mass to be maintained in contact with a mucosal area of the mammal. The mass contains an active ingredient. Active ingredients include psilocin or a prodrug of psilocin. Menthol, L-arginine, and the active ingredient are distributed in the water-soluble mass.

### BACKGROUND

### 1. Field of the Disclosure

The present disclosure relates to compositions and methods for delivering psilocin to a mammal. In particular, the disclosure relates a thin film comprising menthol, L-arginine, and psilocin or a prodrug thereof, for efficient delivery to a mammal. The thin film may be a mucoadhesive strip. The disclosure also relates to a method for treating a human by microdosing with psilocin.

### 2. Description of Related Art

Psilocin is an active psychotropic drug. A common source of psilocin is ‘magic mushrooms’ and other products. Typically, magic mushrooms and other sources for psilocin contain a prodrug of psilocin. The prodrugs are converted to psilocin in the body of a mammalian user.

Psilocin is the topic of research directed to treatment of disease, sickness, and other disorders found in mammals. These disorders may include depression, obsessive/compulsive disorder, smoking, cocaine addiction, cancer-related or other end-of-life psychological distress, and cluster headaches. Psilocin also is also used recreationally to produce altered states of consciousness and to produce mystical experiences.

Psilocin typically is often introduced to a mammalian body by ingestion of a food or beverage containing psilocin or a prodrug thereof. For example, fungal fruiting bodies, whether dried or fresh, can be eaten or added to other foods. An example of a prodrug containing beverage is herbal tea. However, these methods deliver inconsistent amounts of the active ingredient or prodrug of the ingredient.

There is a need in the art for a system and method that delivers a consistent dosing amount of psilocin or a prodrug thereof, in order to treat a disorder of the human body.

### SUMMARY OF THE DISCLOSURE

The disclosure is directed to compositions and methods for dosing and delivery of psilocin and prodrugs thereof.

In one aspect, the disclosure provides a system for delivering a dosing amount of psilocin or a prodrug of psilocin to a mammal, the system comprising: a dissolvable mucoadhesive mass adapted to be maintained in adhesive contact with a mucosal area of a mammal; at least a dosing amount of an active ingredient selected from the group consisting of psilocin, a prodrug of psilocin, and blends thereof; wherein menthol, L-arginine, and the at least a dosing amount of the active ingredient are distributed in the dissolvable mucoadhesive mass.

In another aspect, the disclosure provides a method for delivering a dosing amount of psilocin or a prodrug of psilocin to a mammal, the method comprising: providing to the mammal a dissolvable mucoadhesive mass adapted to be maintained in adhesive contact with mucosal area of the mammal; wherein the dissolvable mucoadhesive mass includes menthol, L-arginine, and at least a dosing amount of an active ingredient selected from the group consisting of psilocin, a prodrug of psilocin, and blends thereof; and maintaining the dissolvable mucoadhesive mass in adhesive contact with mucosal area of the mammal to deliver the dosing amount through the mucosal area.

In still another aspect, the disclosure provides a method for a method for treating a human in need of treatment for a disorder, the method comprising delivering a dosing amount of psilocin or a prodrug of psilocin to the human, the method comprising: providing to the human a water-soluble mass adapted to be maintained in contact with a mucosal area of the human; wherein the water-soluble mass includes menthol, L-arginine, and at least a dosing amount of an active ingredient selected from the group consisting of psilocin, a prodrug of psilocin, and blends thereof; and maintaining the water-soluble mass in contact with mucosal area of the human to deliver the dosing amount through the mucosal area.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a depiction of an embodiment of a of the disclosure; and

FIG. 2 is a depiction of another embodiment of the disclosure.

### DETAILED DESCRIPTION

In accordance with one aspect, the disclosure provides a system for delivering a dosing amount of psilocin or a prodrug of psilocin to a mammal. The system comprises a water-soluble mass adapted to be maintained in contact with a mucosal area of the mammal. The system includes at least a dosing amount of active ingredients selected from the group consisting of psilocin, a prodrug of psilocin, and blends thereof. The system also includes menthol and L-arginine, which are distributed in the water-soluble mass together with the psilocin or prodrug thereof. The dosing amount of active ingredients may be precisely controlled because the system reliably delivers a known amount of active ingredient.

The following definitions and abbreviations are used for convenience throughout the specification.

Microdosing is providing a dose of a drug or a prodrug that is too small to produce a perceptible cognitive effect. Typically, a microdose may be between about 3 percent and about 20 percent of a standard dose.

Psilocin is the active metabolite of psilocybin.

Psilocybin is the prodrug present in selected mushrooms. These mushrooms may be known as “magic mushrooms.” Psilocybin is absorbed and metabolized into the active drug, psilocin.

Sub-perceptional, as it related to dosing, is dosing at a level at which the user does not perceive effects or hallucinations, such as visual, auditory, or bodily perceptions, such as those perceived by a user of at least a standard dose.

5-HT2a—a specific serotonin receptor.

5-HT2c—a specific serotonin receptor.

fMRI—functional magnetic resonance imaging.

LSD—Lysergic acid diethylamide.

MAPS—Multidisciplinary Association for Psychedelic Studies.

MDMA—3,4 methylenedioxymethamphetamine.

OCD—obsessive compulsive disorder.

PTSD—post-traumatic stress disorder.

RCT—randomized clinical trial.

Psilocin at full or standard dose is the subject of medical research directed to treatment of disorders such as disease, sickness, and harmful behavior found in mammals. Psilocin also is used recreationally to produce psychedelic effects. The FDA has called psilocybin a ‘breakthrough’ therapy for treatment resistant major depression. This means the FDA expects psilocybin to replace the current treatment for treatment resistant depression. This characterization is based on full dosing, as there is very little research on microdosing psilocybin. Furthermore, the legal status of psilocybin and hallucinogenic mushrooms in almost all countries of the world has made it almost impossible to conduct serious, well-designed studies on the topic. However, several US states and Canada have passed legislation that now permits or soon will permit medical research, such as clinical trials, on psilocybin, psilocin and psychedelic mushrooms.

Psilocybin is a prodrug of psilocin. As a prodrug, psilocybin is converted to psilocin in the mammal body. Psilocybin typically is obtained from mushrooms of the Basidiomycota species. In particular, hallucinogenic mushrooms are found in the Psilocybe, Gymnopilus, Panaeolus, Copelandia, Hypholoma, Pluteus, Inocybe, Conocybe, Panaeolina, Gerronema, Galerina, and other genera. Psilocin and psilocybin also can be produced synthetically. The concentrations of psilocin and psilocybin, as determined by high-performance liquid chromatography, are in the range of 0.14-0.42% and 0.37-1.30% (dry weight) in the whole mushroom, 0.17-0.78% and 0.44-1.35% in the cap, and 0.09-0.30%/0.05-1.27% in the stem, respectively.

Psilocin is not the only psilocybin derivative in “magic” mushrooms. Other derivatives include norpsilocin, baeocystin, norbaeocystin, and aeruginascin. The derivatives and amounts present vary in different parts of the mushroom. They also vary in between species and even within batches of the same species. There is little research on these other minor psilocybin derivatives.

Psilocybin and psilocin are tryptamine alkaloids and structural analogs of the neurotransmitter serotonin. Psilocybin is a prodrug of psilocin. As the skilled practitioner recognizes, a prodrug is not active in prodrug form. However, in the body, psilocybin prodrug is quickly converted in the liver by enzymatic dephosphorylation into psilocin active by alkaline phosphatase and other esterase enzymes. Without the phosphate group, psilocin becomes more lipid soluble than psilocybin. The increased lipid solubility makes psilocin metabolically available in the body. Psilocin also is able to cross the blood brain barrier and is more easily absorbed in the intestines.

Psilocybin and psilocin reach the human blood plasma in 20-40 minutes after oral administration. Maximum levels occur in the blood 80-105 minutes after oral administration. About 80% of metabolized psilocybin gets excreted in the urine as a compound called psilocin-Some psilocin and psilocybin (only about 3-10%) are excreted in the urine, mostly in a conjugated form with glucoronic acid. This last is then further metabolized, psilocin-being the main urinary metabolite. A study of escalating doses of psilocybin revealed that all of the psilocybin is converted to metabolites. The pharmacokinetics of the psilocin were linear with a half-life of three hours.

In the United States, psilocybin is a Schedule I substance under the Controlled Substances Act, meaning that it has a high potential for abuse, no currently accepted medical use in treatment in the United States, and a lack of accepted safety for use, even under medical supervision. Psilocybin also is controlled in other countries and by international cooperation bodies such as the United Nations.

However, the effects of microdosing of psilocin are the subject of medical research. For example, use of microdosing of psilocin over several weeks, with or without standard psychotherapy sessions, is being tested in support of this application to determine whether psilocin can be safely used to achieve therapeutic effects.

Anecdotal reports suggest that microdosing enhances well-being and cognition. Microdosing of psilocin may reduce reported levels of depression and stress; may lower levels of distractibility; may increase absorption; and may increase neuroticism. Preliminary information discloses a direct, dose related effect that lasts about 24 hours and suggests an additional cumulative effect of regular ingestion of doses over a long period. These and additional effects may be the subjects of additional study. Microdosing of psilocin may therefore be used to treat one or more human disorders.

Menthol is a mucous membrane permeation enhancer that has flavoring and anesthetic properties. In particular, menthol also produces a local analgesic or anesthetic effect on mucosa. As a mucosal penetrant, menthol serves to increase or enhance penetration of both L-arginine and the active ingredient into the submucosal tissues.

L-arginine is a biological precursor of nitric oxide. Nitric oxide is the only substrate for the Nitric Oxide Synthase pathway that converts L-arginine into nitric oxide, a potent vasodilator. Nitric oxide is produced by three isoforms of NO Synthase, NOS. The three isoforms, or isoenzymes, of Nitric Oxide Synthase, are eNOS (endothelial-cells that line the blood vessels), nNOS (neuronal) and iNOS (inducible). Although eNOS has the greatest contribution to vasodilation, all three isoenzymes contribute to vasodilation of sub-mucous membrane blood vessels. In addition, the nitric oxide stimulates the production of cyclic GMP (cGMP, guanosine mono phosphate) a very potent and long acting vasodilator. Vasodilation encourages distribution of the active ingredients throughout the body.

Broadly, there are four typical methods for administering an active agent to a mammal that do not involve an injection. An active agent can be applied topically, can be inhaled, can be consumed orally, or can be administered by sublingual/buccal absorption. Each of these methods has unique benefits and potential drawbacks. Neither topical application nor inhalation delivery is a preferred delivery method for psilocybin and psilocin. For example, inhalation may cause irritation of the trachea and lungs.

Oral consumption of psilocybin and psilocin is wasteful. These active ingredients are metabolized in the liver. Therefore, oral consumption can lead to deactivation of psilocin by way of first pass metabolism. First pass metabolism, also known as first pass effect or presystemic metabolism, reduces the concentration and, therefore, the bioavailability of a drug before the drug reaches the circulatory system. Metabolism occurs in the gut and the liver.

Mucosal delivery avoids first pass metabolism. In embodiments of the disclosure, mucosal delivery makes use of a water-soluble mass adapted to be maintained in contact with a mucosal area. In embodiments, the active ingredient is in the mass. The mass is shaped to facilitate contact with the oral mucosa.

In some embodiments of the disclosure, the mass may be shaped into thin strips that are inserted into the buccal pouch, a space generally defined between a cheek and the gums. In other embodiments, the mass may be shaped to be held under the tongue, effecting drug release into and through the oral mucosa and minimizing release of the active ingredients into the gastrointestinal tract, thereby bypassing gastrointestinal and hepatic “first pass” metabolism processes. The water-soluble mass is shaped to avoid discomfort of the mammal and does not introduce an objectionable mouth feel, such as a bitter flavor or a gritty or any other undesirable feeling in the mouth.

In embodiments, the water-soluble mass is shaped to be maintained in contact with mucosa. In some embodiments, the water-soluble mass is shaped to maximize contact with the mucosa and to minimize loss of active ingredient to first pass metabolism of active ingredient carried to the gut and liver by saliva during administration of the active ingredient.

In embodiments of the disclosure, the shape of the water-soluble mass is adapted to maintain contact with the mucosa. To maintain contact with the mucosa and provide an unobjectionable mouthfeel, the mass may be formed of water-soluble material that is shapable or pliable. Thus, in some embodiments, the mass may be shaped as a thin layer, such as a film or strip. In other embodiments, the water-soluble mass may have a shape such as a pastille or troche. In some embodiments, a pastille or troche may be made from polymeric carrier matrix materials such as starches and gum Arabic, which bind the active ingredients in a hydrocolloidal matrix. The matrix helps control the delivery speed of the active ingredient as the matrix dissolves. In some embodiments, the degree of cross linking may be adjusted to have a soft, easily dissolved form that delivers active ingredient relatively quickly. In other embodiments, a high degree of cross linking may provide a firmer water-soluble mass that remains in the mouth for a longer time and thus delivers active ingredient more slowly. The skilled practitioner will know how to obtain products having a desired dissolution rate in accordance with the disclosure herein.

In embodiments, the water-soluble mass is a matrix that dissolves in the mouth. The mass may be formed into any suitable shape, such as a parallelepiped, particularly one in the form of a thin strip. The mass also may be in the form of a cylinder or prism. Because the mass may be thin, it may useful to describe the shapes as rectangular, square, triangular, and the like. Triangular shapes may be especially suitable for insertion between the gums and the cheek.

In some embodiments, a fast-dissolving drug delivery system is used to quickly administer the active ingredients. Such films comprise water-soluble polymers as the polymeric carrier matrix, such as pectin; gelatin; sodium alginate; hydroxypropylcellulose, polyvinylalcohol, maltodextrins, grades of methyl cellulose; grades of carboxymethylcelluloses; pullulan and other polysaccharides; surfactants, such as sodium lauryl sulfate and the Tween® products; and may include plasticizers, sweeteners, colors, and the like.

In some embodiments, mucoadhesive polymers have numerous hydrophilic groups, such as hydroxyl, carboxyl, amide, and sulfate groups in the structure. These hydrophilic groups attach to mucus membranes by various interactions such as hydrogen bonding and hydrophobic or electrostatic interactions. In some embodiments, these hydrophilic groups also cause polymers to swell in water and, thus, expose the maximum number of adhesive sites. The extended retention time results in enhanced bioavailability and maximum drug concentration in the plasma and decreased time until maximum concentration of the drug in the plasma.

In some embodiments, the mucoadhesive embeds into irregularities of the mucosal surface. In some embodiments, electrostatic mechanisms may result in transfer of electrons across the adhesive interface that creates a concentration gradient and thus a more attractive region for diffusion of the drug. Stronger adhesion to the mucous membrane by the mucoadhesive is associated with superior absorption.

Water-soluble materials suitable for forming the water-soluble mass are commercially available. The ratio of components may be adjusted to form a water-soluble mass having selected desired dissolution rates.

In embodiments, menthol acts as a permeation enhancer that enhances or improves the diffusion of L-arginine and active ingredients across the oral mucus membrane barrier. Menthol functions as a permeation enhancer through vasodilation for a short duration, i.e. about 1 minute to 5 minutes. Use of L-arginine in embodiments of the disclosure induces the nitric oxide synthase enzyme to produce nitric oxide and cyclic GMP, which induces prolonged vasodilation. The rate limiting factor of the induction of the nitric oxide synthase enzyme is the availability of L-arginine. By extending the 1-5 minutes of vasodilation provided by menthol to about 20-40 minutes, L-arginine can provide an extended period for absorption into the oral mucosal vasculature.

In some embodiments, menthol functions as a mucus membrane permeation enhancer which causes transient vasodilation and allows the L-arginine and active ingredients to enter the oral mucosa easily and rapidly. The absorbed L-arginine then induces production of nitrous oxide in oral mucosal cells, which diffuses to neighbor cells and reaches its target, guanylate cyclase. The activation of guanylate cyclase induces an increase in cyclic guanylate monophosphate, which is a signaling messenger that relaxes smooth muscle tissues and leads to vasodilation and increased blood flow. In embodiments, the prolonged blood flow resulting from the combination of menthol and L-arginine provides enhanced pharmacokinetic bioavailability of the active ingredients.

Therefore, in various embodiments, menthol may be present in the mucoadhesive mass in an amount that is sufficient to cause vasodilation in the user for at least 1 minute. In other embodiments, menthol may be present in the mucoadhesive mass in an amount that is sufficient to cause vasodilation for at least 3 minutes, or at least 5 minutes. By weight in the mucoadhesive mass, menthol may be present in an amount of from about 3 mgs to about 8 mgs—or, about 5 mgs.

Furthermore, in various embodiments, L-arginine may be present in the mucoadhesive mass in an amount that is sufficient to cause prolongation of vasodilation in the user for at least 20 minutes. In other embodiments, L-arginine may be present in the mucoadhesive mass in an amount that is sufficient to cause prolongation of vasodilation in the user for at least 30 minutes, or at least 40 minutes. By weight in the mucoadhesive mass, L-arginine may be may be present in an amount of from about 6 mgs to about 12 mgs.—or, about 9 mgs.

As described above, in embodiments, the dissolution rate of the water-soluble mass is adjusted to provide a period sufficient to maximize delivery of the active ingredients while minimizing loss of active ingredients to ingestion. Embodiments may have different loss rates for active ingredients, typically depending upon the resistance of the water-soluble mass to dissolution and thus length of time in the mouth. Therefore, in embodiments of the disclosure, the amount of active ingredient present may be increased from the therapeutic or dosing amount to compensate for such losses. Thus, at least a dosing amount of active ingredient may be included in the water-soluble mass. In some embodiments, delivery rate is controlled by controlling the concentration of active ingredient in the delivery layer.

Embodiments of the disclosure provide a system for delivering a well-controlled dosing amount of active ingredient. However, in other embodiments, delivery precision is further increased in another aspect of the disclosure. Embodiments provide a system for delivering a dosing amount of psilocin or a prodrug of psilocin to a mammal includes a dissolvable mucoadhesive mass. In embodiments, the mucoadhesive mass maintains adhesive contact with a mucosal area of the mammal and includes at least a dosing amount of an active ingredient selected from the group consisting of psilocin or a prodrug of psilocin. Menthol, L-arginine, and the at least a dosing amount are distributed in the dissolvable mucoadhesive mass.

In some embodiments, dosage forms include orally dissolving mucoadhesive films or strips made of a water-soluble mass formed from a mucoadhesive polymeric carrier matrix including active ingredient, menthol, and L-arginine. In some embodiments, film or strips are flexible, quickly wettable, and non-irritating to the user. The strips dissolve quickly while providing an adequate level of mucoadhesion. For the present invention, it is preferable to use films that provide a quick enough dissolution rate, most desirably between about 1 minute and about 20 minutes, while providing an acceptable mucoadhesion level such that the film is not easily removable once it is placed in the oral cavity of the user.

In embodiments, the terms “mucoadhesion” and “mucoadhesive” refer to the same adhesive or adherent property or effect in which a product binds to the mucin layer of a biological membrane, such as the oral mucosa. Mucoadhesion is associated with benefits such as controlled, sustained release; prolonged residence time at the site of action; the ability to target a specific area or region of a selected mucosal surface; and ease of application which leads to higher rates of patient compliance. For example, a mucoadhesive strip according to embodiments of the disclosure can be a sheet or film that adheres to the mucosal surface of the mouth and is difficult to remove once placed in the mouth. The adherence helps achieve optimum absorption of the pharmaceutically active ingredient. In embodiments, a strip that is sufficiently adherent to oral mucosa will not become dislodged by typical mouth manipulations, such as talking or swallowing. In some embodiments, the adhesion level or degree will be sufficient to preclude dislodgment by motion of the tongue, including motions intended to dislodge the strip.

In embodiments of the disclosure, the terms “strip” and “film” refer to typically thin films. In embodiments, strips or films suitable for use on oral mucosa may have a flexibility that enables placement of the strip into the oral cavity of the user. Films may be in a single layer or they may be multi-layered, including laminated films. In embodiments, flexibility may be adjusted by the thickness of the strip or film or by the stiffness of the strip or film. In embodiments, layers may be the same size. In some embodiments, a non-delivery layer may be larger than a delivery layer (i.e. have a larger thickness). Specifically, the film may comprise a first layer containing the active ingredient where the first layer has a first thickness, and then the film may further comprise a second layer that is devoid of the active ingredient having a second thickness that is larger than the first thickness.

In such embodiments, the non-delivery layer covers all sides of the delivery layer except the side in contact with the mucosa. In such embodiments, less active ingredient may be lost to the saliva. In other embodiments, non-delivery layers may not cover the entirety of the delivery layer because to do so may cause the system may not be comfortable in the mouth. In some embodiments, layers may have different thicknesses. In other embodiments, the layers may have the same thickness.

Mucoadhesive strips are generally known in the art. In embodiments of the disclosure, matrix for use in strips or films can include a synthetic polymer such as, but not limited to, polyacrylic acid; polyethylene oxide, such as a suitable grade of Polyox™, available from Dupont, USA; polymethacrylate derivatives; polycarbophil; poloxamer mixtures; Carbopol® polymers, available from Lubrizol, USA; hydroxy-methylcellulose; hydroxy-propylcellulose; hydroxypropylmethyl-cellulose (HPMC); polyethylene glycol (PEG); as well as naturally occurring polymers such as hyaluronic acid and chitosan, alone or in combination. Many of these ingredients are available in compositions that have selected properties, such as molecular weight, proportion of components, and the like. Thus, properties and characteristics of the layers can be adjusted for flexibility, solubility, active ingredient flow rate to the mucosa, and the like by selection of components for the compositions.

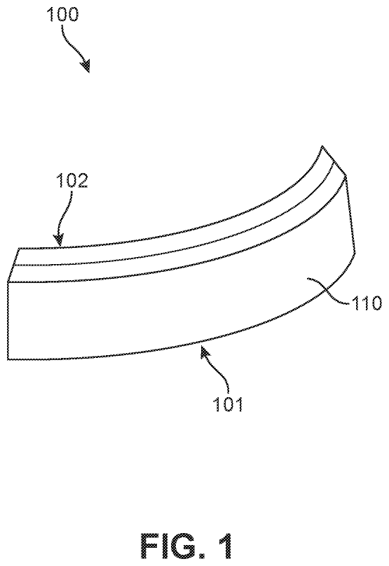

FIG. 1 depicts laminated strip or film 100. Mucosal layer 101 forms the mucosal side of laminated strip 100. Lingual layer 102 laminated thereto forms the lingual side of laminated strip. Lingual layer 102 also may be in contact with the teeth. Surface 110 is the mucosally adherent surface through which active ingredient is delivered. Strip 100 may be made of any number of layers. The properties and characteristics of these layers may be selected to provide a strip having desired flexibility, amount of active ingredient delivered, and mucosal adhesion, for example. In embodiments of the disclosure, the strip may be formed of a single layer. In such embodiments, delivery of active ingredient occurs on all sides. Thus, such a strip is particularly suitable for sublingual application.

In embodiments of the disclosure, a first layer is the layer from which the active ingredient is delivered to the mucosa. In embodiments of a strip having solely a layer containing active ingredient for mucosal delivery, the properties and characteristics of the layer can be selected to ensure that the entire strip dissolves while delivering active ingredient to the mucosa. In some embodiments, the strip may contain two layers, a mucosal or delivery layer and a second layer. In some embodiments, the second layer may be designed to last longer than and protect the active ingredient delivery layer. In this way, loss of active ingredient to swallowed saliva is minimized. This second layer further may be designed to enclose the delivery layer on all sides except for the buccal contact surface. In some embodiments, third and additional layers may be added on the lingual side of the mucosal or delivery layer.

In some embodiments, a layer may contain flavorants, colorants, odorants, and other additives that may improve the mammal's tolerance of the strip. Some embodiments of the disclosure may contain compositions, such as bitterants, in concentrations or forms that will not be objectionable if the strip is allowed to dissolve, but that will impart an unpleasant taste or sensation if the strip is chewed or is scraped against the teeth. In embodiments, such compositions are crystalline but slowly soluble in saliva, so that chewing the strip would be unpleasant, but dissolution of the crystals would be unobjectionable. In another embodiment, the objectionable composition is a liquid or a solid encapsulated so that the capsule is broken only by chewing, and the encapsulation remains unopened during the period of use.

In some embodiments, the system described herein may be used for microdosing. As used herein, microdosing relates to delivery of a sub-hallucinogenic quantity of active ingredient. Typically, a microdose is between about 3 percent and about 25 percent of a typical hallucinogenic dose, more typically between about 5 percent and about 20 percent more typically between about 5 percent and about 15 percent of a typical hallucinogenic dose. Even more typically, a microdose is between about 5 percent and about 10 percent of a typical dose. For “magic” mushrooms, a hallucinogenic dose is about 3 grams of mushrooms.

The concentration of psilocin in the whole mushroom may be in the range of between about 0.14 weight percent and about 0.42 weight percent, based on the dry weight of the whole mushroom. The concentration of psilocin in the cap may be between about 0.17 weight percent and about 0.78 weight percent, based on the dry weight of the cap. The concentration of psilocin in the stem is between about 0.09 weight percent and about 0.30 weight percent, based on the weight of the stem.

The concentration of psilocybin in the whole mushroom may be in the range of between about 0.37 weight percent and about 1.30 weight percent, based on the dry weight of the whole mushroom. The concentration of psilocybin in the cap may be between about 0.44 weight percent and about 1.35 weight percent, based on the dry weight of the cap. The concentration of psilocybin in the stem is between about 0.05 weight percent and about 1.27 weight percent, based on the weight of the stem, respectively.

In embodiments of the disclosure, a dosing amount of psilocybin-containing mushrooms is in the range of between about 0.02 grams and about 0.6 grams, typically between about 0.05 grams and about 0.45 grams, and more typically between about 0.1 grams and about 0.3 grams. Standardized or fully analyzed extracts from the mushrooms may be utilized to maintain consistency of dose.

More precise dosing can be obtained by using psilocin rather than the prodrug. Extraction of psilocybin from the mushroom in water at about 40° C. or less will not convert psilocybin to psilocin. However, extraction at higher temperatures will dephosphorylate psilocybin and form psilocin.

Some active ingredient may be lost to saliva. In embodiments, some active ingredient may not be delivered from the delivery layer. Therefore, more than the dosing amount may be present in the delivery layer to accommodate this loss or delivery failure. Estimates of the loss may be made in embodiments and the dosing amount increased to compensate.

In embodiments of the disclosure, menthol is a lipophilic mucus membrane permeation enhancer that improves the diffusion of L-arginine and active ingredient across the oral mucus membrane barrier. Menthol also aids in the absorption of active ingredient across the oral mucosa. Menthol functions as a permeation enhancer for a short period. In some embodiments, menthol may serve as a permeation enhancer for a period between about 1 minute to about 5 minutes. The use of L-arginine in embodiments of the disclosure induces the nitric oxide synthase enzyme to produce nitric oxide and cyclic GMP, as disclosed above. This effect induces prolonged vasodilation. The rate limiting factor of the induction of the nitric oxide synthase enzyme is the availability of L-arginine. In embodiments, L-arginine may extend the period for absorption of the active ingredient into the oral mucosal vasculature up to about 60 minutes, typically to between about 10 minutes and about 50 minutes, and more typically to between about 20 minutes and about 40 minutes.

In some embodiments, menthol in the delivery layer functions as a mucus membrane permeation enhancer that causes transient vasodilation and allows the L-arginine and active ingredient to enter the oral mucus membranes easily and rapidly. In embodiments of the disclosure, the absorbed L-arginine then induces production of nitrous oxide in oral mucosal cells, which diffuses to neighbor cells and reaches its target, guanylate cyclase. Activation of guanylate cyclase induces an increase in cyclic guanylate monophosphate, which is a signaling messenger that relaxes smooth muscle tissues and leads to vasodilation and increased blood flow. The vasodilation is essentially immediate. The increased and prolonged blood flow provided by the combination of menthol and L-arginine provides enhanced pharmacokinetic bioavailability of the active ingredient.

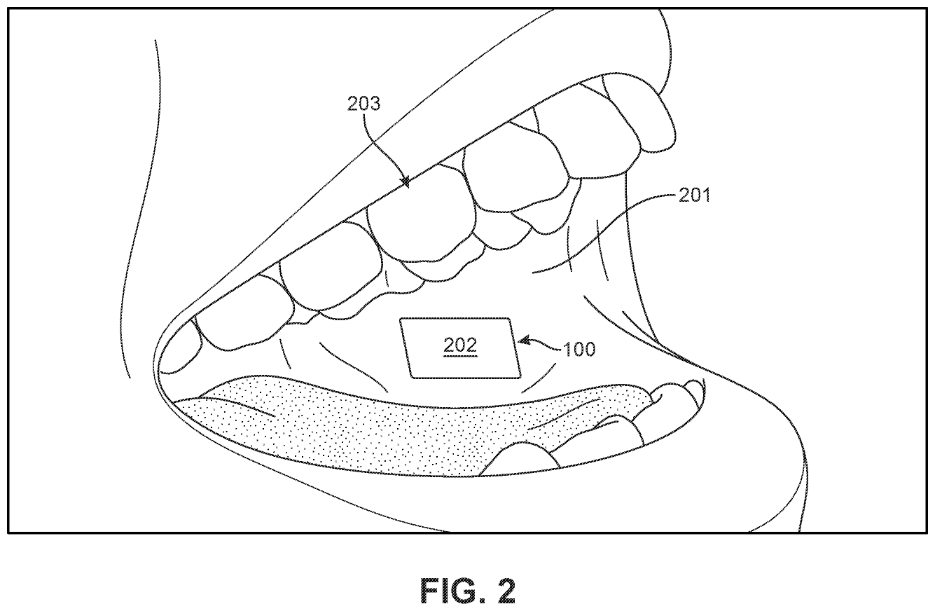

In other embodiments, the disclosure provides a method for delivering a dosing amount of psilocin or a prodrug thereof to a mammal. In accordance with embodiments of the disclosure, the method provides a water-soluble mass to the mammal. The mass is maintained in contact with mucosal area of the mammal. FIG. 2 illustrates an embodiment of a method in accordance with the disclosure. Laminated strip 100 is in place on cheek 201 of a user. Lingual layer 202 is adjacent teeth 203. The mass includes menthol, L-arginine, and at least a dosing amount of the psilocin, a prodrug of psilocin, and blends thereof. In accordance with the method, the water-soluble mass is maintained in contact with mucosal area of the mammal to deliver the dosing amount through the mucosal area.

In embodiments of the disclosure, the mass dissolves essentially completely while delivering the active ingredient. In embodiments of the disclosure, a layer not involved in delivery of active ingredient dissolves completely. In other embodiments of the disclosure, a layer not involved in delivery of active ingredient does not dissolve completely. In such embodiments, a layer that does not dissolve completely may be swallowed or may be removed from the oral cavity through the mouth.

In embodiments, the method for delivering a dosing amount of psilocin or a prodrug of psilocin to a mammal is carried out by provided to the mammal a dissolvable mucoadhesive mass adapted to be maintained in adhesive contact with mucosal area of a mammal. The dissolvable mucoadhesive mass includes menthol, L-arginine, and at least a dosing amount of psilocin or a prodrug of psilocin. The dissolvable mucoadhesive mass is maintained in adhesive contact with mucosal area of the mammal to deliver the dosing amount through the mucosal area.

In embodiments of the disclosure, the mass dissolves completely while delivering the active ingredient. In embodiments of the disclosure, any layer not delivering active ingredient may then be released from the mucosa. In such embodiments, this release may be indicative of completed delivery of the active ingredient. In embodiments of the disclosure, a layer not involved in delivery of active ingredient dissolves completely. In other embodiments of the disclosure, a layer not involved in delivery of active ingredient does not dissolve completely. In such embodiments, a layer that does not dissolve completely may be swallowed or may be removed from the oral cavity through the mouth.

In embodiments, a microdose of between about 5 percent and 10 percent of a typical dose of psilocybin is used to produce health benefits in humans. In some embodiments, the health benefits relate to levels of anxiety, depression, and stress (ADS), and to obsessive-compulsive disorder symptoms. Any reliable test may be used. In embodiments, ADS can be characterized or measured by the DASS-21 test, and OCD can be characterized by the Yale-Brown Obsessive Compulsive Scale.

In embodiments of the disclosure, a microdose may be in the range of between about 0.01 mg/kg and about 0.1 mg/kg of active ingredient per kg of the body weight of the user; between about 0.02 mg/kg and about 0.09 kg/mg; between about 0.03 mg/kg and about 0.08 mg/kg; between about 0.04 mg/kg and about 0.07 mg/kg; and between about 0.05 mg/kg and about 0.06 mg/kg.

In embodiments of the disclosure, a macrodose may be in the range of between about 0.3 mg/kg to about 0.6 mg/kg of active ingredient per kg of body weight of the user.

Psilocin is the active drug that has the therapeutic effects via specific serotonin receptors in the brain. Psilocin has a high binding affinity with 5-HT2a and 5-HT2c serotonin receptors. These serotonin receptors in the cortex of the brain are primarily for mood, cognition, sleeping, eating, and memory. Research suggests that psilocybin, and other psychedelics, bind to the serotonin receptors and in the cortical centers of the brain, it overstimulates these receptors resulting in the formation of new neural connections. Studies suggest that there may also be an indirect dopaminergic therapeutic effect through dopamine receptors as well.

The mushroom contains both the prodrug, psilocybin, and the active drug, psilocin, in lesser amounts. Psilocin has several dose dependent effects that include euphoria, visual and auditory hallucinations, changes in perception, and a distorted sense of time. Interestingly growing evidence suggests that regional alterations in glutamate is associated with the ‘ego dissolution’ seen with psilocin. LSD and psilocin indirectly affect glutamatergic neurotransmission through recruitment of N-methyl-D-aspartate (NMDA) receptors. Research supports this effect on regional glutamate levels as the neurochemical basis for the therapeutics effects of psilocin. Similar effects can be seen with LSD, mescaline, and N.N-Dimethyltryptamine (DMT).

A study (6) of brain imaging with use of psilocybin was conducted utilizing an ultra-high field multimodal brain imaging approach and demonstrated that psilocybin (0.17 mg/kg) induced region-dependent alterations in glutamate, which predicted distortions in the subjective experience of one's self (ego dissolution). Whereas higher levels of medial prefrontal cortical glutamate were associated with negatively experienced ego dissolution, lower levels in hippocampal glutamate were associated with positively experienced ego dissolution. Such findings provide further insights into the underlying neurobiological mechanisms of the psychedelic, as well as the baseline state.

Psilocin doses usually last two to six hours, but may last much longer. Tolerance to psilocin builds up and dissipates quickly. Using psilocin more than twice a week can lead to diminished effects. A cross-tolerance can develop between psilocin and LSD or mescaline.

Oral doses of psilocybin—0.3 mg/kg, 0.45 mg/kg, and 0.6 mg/kg—were given to 12 healthy adults at monthly intervals. The fixed doses using the pharmacokinetic parameters suggest that an oral dose of 25 mg should approximate the drug exposure of a 0.3 mg/kg oral dose of psilocybin. Doses of 0.6 mg/kg and higher are not therapeutic doses. However, no serious physical or psychological events occurred during or within 30 days of any dose.

In embodiments of the disclosure, dosing of a human with precise quantities of active drug or prodrug achieves a reduction of indicia of illness, essentially without adverse effects. Dosing of the quantities described in the disclosure may be given on alternate days, every third day, or every fourth day, for example. Such dosing schedules reduces the likelihood of developing a tolerance to the psilocin.

### EXAMPLES

Efficacy of embodiments of the disclosure is illustrated by the following examples. After a two-week period of using placebo buccal strips, human subjects are provided doses of psilocin at a low, first level for two weeks. The psilocin is from extracts from mushrooms to precisely control the dose. A one-week wash-out period follows the first dosing period. Then, a second period of dosing at a higher, second level of psilocin for two weeks.

The examples thus are prepared as part of an eight-week, double-blind, randomized, placebo controlled, cross-over design study to evaluate the effectiveness of two different microdoses of psilocybin on levels of anxiety/depression/stress and obsessive-compulsive symptoms in healthy adults. The adults do not have a current psychiatric diagnosis and not taking psychiatric or ‘mind-altering’ drugs, and without contraindications to use of hallucinogenics.

One hundred subjects—100 healthy adults—will be screened for a history of psychological or psychiatric conditions, recent use of medications, and potentially disqualifying conditions by health care providers, prior to acceptance into the study.

The dosing schedule will provide psilocin every third day. This dosing schedule is intended to reduce the chance of a user developing tolerance in view of the fact that the subject's serotonin receptors are rapidly down-regulated by psilocin.

The low dose of active will be 4 mg of psilocybin/psilocin (approximately 0.05 mg/kg for 80 kg adult) and the high dose will be 12 mg of psilocybin/psilocin (approximately 0.15 mg/kg for an 80 kg adult).

Testing for Effect—The standardized and widely accepted DASS-21 anxiety/depression/stress scoring test will be administered by an investigator on day zero of the study, prior to the first dose, then once each week, for a total of six DASS-21 scores during the trial. The Yale-Brown Obsessive Compulsive Scale (YBOCS) will be obtained weekly as well. The investigators will focus the DASS-21 questions so that the subject is providing answers for the “past week.”

Adverse effect monitoring—The subjects will be provided with an investigator contact to contact with questions about the study drug, and will be contacted weekly to answer questions regarding potential adverse effects, as well as hallucinogenic effects from the use of the micro-dose of the drug.

Systems for tracking symptoms, perceived benefits, and perceived adverse or hallucinogenic effects will be in place. The Hallucinogen Rating Scale will administered at 8 hours after the first dose of each of the three legs of the study.

Several steps and measurements will be used weekly to validate that the subject is taking the prescribed amount of the test drug. The study will be designed to ensure that the test subjects are dosing correctly. It is expected that some subjects will attempt to take larger doses than planned or may attempt to share the drug with others.

The study will have 100 subjects with a cross-over designed and a placebo period. This type of study design is expected to increase the power of the study to clearly identify any potential benefits from ‘placebo effect.’ Biostatistical analysis of the results will compare the two doses to each other and to placebo.

Factors such as cannabis use, prior or recent psilocybin use, alcohol consumption, and recent psychological trauma will be factored into subject selection to decrease the chance of confounding of the findings.

While various embodiments of the disclosure have been described, the description is intended to be exemplary, rather than limiting and it will be apparent to those of ordinary skill in the art that many more embodiments and implementations are possible that are within the scope of the disclosure. Accordingly, the disclosure is not to be restricted except in light of the attached claims and their equivalents. Also, various modifications and changes may be made within the scope of the attached claims.

We claim:

1. A system for delivering a dosing amount of psilocin or a prodrug of psilocin to a mammal, the system comprising:
a dissolvable mucoadhesive mass adapted to be maintained in adhesive contact with a mucosal area of a mammal;
at least a dosing amount of an active ingredient selected from the group consisting of psilocin, a prodrug of psilocin, and blends thereof; wherein
menthol, L-arginine, and the at least a dosing amount of the active ingredient are distributed in the dissolvable mucoadhesive mass.

2. The system of claim 1, wherein the dosing amount is a microdose, such that the mucoadhesive mass includes the active ingredient in an amount sufficient to deliver the dosing amount in a range of between about 0.01 mg/kg and about 0.1 mg/kg per kg of a body weight of the mammal.

3. The system of claim 1, wherein the mucoadhesive mass further comprises colorants, flavorants, odorants, bitterants, and other additives.

4. The system of claim 1, wherein the mucoadhesive mass comprises layers, and at least one layer is devoid of the active ingredient.

5. The system of claim 1, wherein the mucoadhesive mass comprises layers, and the layers have different thicknesses.

6. The system of claim 1, wherein the mucoadhesive mass comprises layers, and the layers have different sizes.

7. The system of claim 1, wherein the dosing amount is between about 0.3 mg/kg to about mg/kg of active ingredient per kg of body weight of the mammal.

8. A method for delivering a dosing amount of psilocin or a prodrug of psilocin to a mammal, the method comprising:
providing to the mammal a dissolvable mucoadhesive mass adapted to be maintained in adhesive contact with mucosal area of the mammal;
wherein the dissolvable mucoadhesive mass includes menthol, L-arginine, and at least a dosing amount of an active ingredient selected from the group consisting of psilocin, a prodrug of psilocin, and blends thereof; and
maintaining the dissolvable mucoadhesive mass in adhesive contact with mucosal area of the mammal to deliver the dosing amount through the mucosal area.

9. The method of claim 8 wherein the dosing amount of the active ingredient in the mucoadhesive is from about 4 mg to about 12 mg.

10. The method of claim 8 wherein the dosing amount is from about 0.05 mg/kg to about mg/kg of active ingredient per kg of body weight of the mammal.

11. The method of claim 8 wherein the menthol is present in the mucoadhesive mass in an amount that is sufficient to cause vasodilation in the mammal for at least 1 minute.

12. The method of claim 8 wherein the L-arginine is present in the mucoadhesive mass in an amount that is sufficient to cause prolongation of vasodilation in the mammal for at least 20 minutes.

13. The method of claim 8 wherein
the dosing amount is from about 0.05 mg/kg to about 0.15 mg/kg of active ingredient per kg of body weight of the mammal;
the menthol is present in the mucoadhesive mass in an amount that is sufficient to cause vasodilation in the mammal for at least 1 minute; and
the L-arginine is present in the mucoadhesive mass in an amount that is sufficient to cause prolongation of vasodilation in the mammal for at least 20 minutes.

14. A method for treating a human in need of treatment for a disorder, the method comprising delivering a dosing amount of psilocin or a prodrug of psilocin to the human, the method comprising:
providing to the human a water-soluble mass adapted to be maintained in contact with a mucosal area of the human;
wherein the water-soluble mass includes menthol, L-arginine, and at least a dosing amount of an active ingredient selected from the group consisting of psilocin, a prodrug of psilocin, and blends thereof; and
maintaining the water-soluble mass in contact with mucosal area of the human to deliver the dosing amount through the mucosal area.

15. The method of claim 14 wherein the method includes
the menthol inducing vasodilation in the human for at least one minute; and
the L-arginine prolonging the vasodilation in the human for at least 20 minutes.

16. The method of claim 14 wherein the dosing amount is between from about 4 mg to about 12 mg of the active ingredient in the water-soluble mass.

17. The method of claim 14 wherein
the water-soluble mass is a multi-layer film;
a first layer of the multi-layer film includes the active ingredient, and the first layer has a first thickness;
a second layer of the multi-layer film is devoid of the active ingredient, and the second layer has a second thickness that is larger than the first thickness;
the second layer covers all sides of the first layer except a side of the first layer that is placed into in contact with the mucosal area of the human.

18. The method of claim 14 wherein the water-soluable mass further includes a polymeric carrier matrix material selected from the group consisting of starches, gum Arabic, water-soluble polymers, pectin, gelatin, sodium alginate, hydroxypropylcellulose, polyvinylalcohol, maltodextrins, methyl cellulose, carboxymethylcelluloses, pullulan, other polysaccharides, and mixtures thereof.

19. The method of claim 14 wherein the method further includes adjusting a dissolution rate of the water-soluble mass to provide a time period sufficient to maximize delivery of the active ingredient while minimizing loss of active ingredient to ingestion, by:
selecting a ratio of polymeric carrier matrix components in the water-soluble mass to arrive at a desired dissolution rate, and
selecting a concentration of active ingredient in the water-soluble mass.

20. The method of claim 14 wherein
the dosing amount is from about 0.05 mg/kg to about 0.15 mg/kg of active ingredient per kg of body weight of the mammal;
the menthol is present in the water-soluble mass in an amount that is sufficient to cause vasodilation in the mammal for at least 1 minute; and
the L-arginine is present in the water-soluble mass in an amount that is sufficient to cause prolongation of vasodilation in the mammal for at least 20 minutes.
